(19) United States
(12) Patent Application Publication
(10) Pub. No.: US 2002/0107640 A1
(43) Pub. Date: Aug. 8, 2002

(54) Methods for determining the true signal of an analyte

(76) Inventors: Trey E. Ideker, Cambridge, MA (US); Vesteinn Thorsson, Seattle, WA (US); Andrew F. Siegel, Seattle, WA (US)

Correspondence Address: CAMPBELL & FLORES LLP, 4370 LA JOLLA VILLAGE DRIVE, 7TH FLOOR, SAN DIEGO, CA, 92122, US

(21) Appl. No.: 09/823,850

(22) Filed: Mar. 30, 2001

Related U.S. Application Data
(60) Provisional application No. 60/248,259, filed on Nov. 14, 2000.
(60) Provisional application No. 60/266,388, filed on Feb. 2, 2001.

(51) Int. Cl. G06F 19/00; G01N 33/48; G01N 33/50; G01N 31/00; G06F 1/00

(52) U.S. Cl. 702/19; 702/25; 702/22; 708/131

(57) ABSTRACT

The invention relates to a method of determining a true signal of an analyte, comprising (a) measuring an observed signal x for one or more analytes, and (b) determining a mean signal (μ) and a system parameter (β) for said analyte that produce enhanced values for a probability likelihood of said observed signal, said observed signal being related to said mean signal by an additive error (δ) and a multiplicative error (ε), wherein said system parameter specifies properties of said additive error (δ) and said multiplicative error (ε).

This application is based on, and claims the benefit of, U.S. Provisional Application No. 60/248,259, filed Nov. 14, 2000, entitled Testing for Differentially-Expressed Genes by Maximum Likelihood Analysis of Microarray Data and claims benefit of, U.S. Provisional Application No. 60/266,388, filed Feb. 2, 2001, entitled Methods for Determining the True Signal of an Analyte, which are incorporated herein by reference.

This invention was made with government support under grant number T32 HG 000-35 awarded by the National Institutes of Health and grant number DE-FG03-98ER62652/A000 awarded by the United States Department of Energy. The United States Government has certain rights in this invention.

### BACKGROUND OF THE INVENTION

The invention relates generally to quantitative expression analysis, and more particularly, to methods for identifying significant differences in gene expression.

Although all cells in the human body contain the same genetic material, the same genes are not active in all of those cells. Alterations in gene expression patterns or in a DNA sequence can have profound effects on biological functions. These variations in gene expression are at the core of altered physiologic and pathologic processes. In the past, determinations of differential gene expression only focused on a few genes at a time. DNA microarrays, devices that consist of thousands of immobilized DNA sequences present on a miniaturized surface, have revolutionized the study of gene expression and are now a staple of biological inquiry into gene expression and genetic variations. Arrays are used to analyze a sample for genotyping or for patterns of gene expression. Using the microarray, it is possible to observe the expression level changes in tens of thousands of genes over multiple conditions, all in a single experiment. Depending on the conditions assayed, differentially-expressed genes may be implicated in cancer, aging, or a metabolic pathway of interest.

Generally, microarrays are prepared by binding DNA sequences to a surface such as a nylon membrane or glass slide at precisely defined locations on a grid. Using an alternate method, some arrays are produced using laser lithographic processes and are referred to as biochips or gene chips. For genotyping analysis, the sample is genomic DNA. For expression analysis, the sample is cDNA, DNA copies of mRNA. The DNA samples are tagged with a radioactive or fluorescent label and applied to the array. Single stranded DNA will bind to a complementary strand of DNA. At positions on the array where the immobilized DNA recognizes a complementary DNA in the sample, binding or hybridization occurs. The labeled sample DNA marks the exact positions on the array where binding occurs, allowing automatic detection. The output consists of a list of hybridization events, indicating the presence or the relative abundance of specific DNA sequences that are present in the sample. DNA array technology provides a method for rapid genotyping, facilitating the diagnosis of diseases for which a gene mutation has been identified as well as for diseases for which known gene expression biomarkers of a pathologic state, or signature genes, exist.

A crucial step in the analysis of expression data is determining which genes are expressed differently between two cell populations. Usually, a gene is said to be “differentially-expressed” if its ratio of expression level in one population to expression level in a second population exceeds a certain threshold. This threshold is set based on the observation that in control experiments where the two cell populations are identical, few if any genes have expression ratios exceeding the threshold. However, it is common knowledge that this approach is imprecise, because the uncertainty in the expression ratio is greater for genes that are expressed at low levels than for those that are highly expressed. More sensitive methods have been employed in a few cases, but development of a general, formal statistical test for identifying differentially-expressed genes has remained an open problem.

Thus, there exists a need for a mathematical model of the variability observed over repeated observations of intensities for biomolecules represented on an array. The present invention satisfies this need and provides related advantages as well.

### SUMMARY OF THE INVENTION

The invention relates to a method of determining a true signal of an analyte, comprising (a) measuring an observed signal x for one or more analytes, and (b) determining a mean signal (μ) and a system parameter (β) for said analyte that produce enhanced values for a probability likelihood of said observed signal, said observed signal being related to said mean signal by an additive error (δ) and a multiplicative error (ε), wherein said system parameter specifies properties of said additive error (δ) and said multiplicative error (ε)

### BRIEF DESCRIPTION OF THE DRAWINGS

FIG. 1 shows the (A) increase of standard deviation; (B) increase of correlation with absolute level of intensity x′ or y′; and (C) normal probability plot for the 80 samples of x′ pertaining to a single, representative gene.

FIG. 2 shows scatter plots of estimated μ_{y} versus μₓ for each gene represented on the whole-yeast genome microarray, for (A) the control experiment YPRG versus YPRG and (B) the YPR versus YPRG comparison, while (C) shows the distribution of four (x,y) pairs for two genes in the YPR versus YPRG comparison.

FIG. 3 shows array images corresponding to hybridizations performed for each of eight controlled GAL80 ratios where four (x,y) intensity measurements per gene were obtained at each controlled GAL80 ratio by using (A) two spots from a forward Cy3:Cy5 labeling scheme and (B) two spots from a reverse Cy5:Cy3 labeling scheme and (C) comparison of each controlled ratio to measured ratio (y/x) for the forward-array (red dots) or reverse-array (green dots).

### DETAILED DESCRIPTION OF THE INVENTION

The invention provides a method of determining relative amounts of an analyte between samples. The invention also provides a method of determining the true signal of an analyte. The method of the invention accounts for multiplicative and additive errors influencing the observed signals for an analyte and estimates system parameters based on the observed signals using maximum likelihood estimation. By presenting an error model and associated significance test, the methods of the invention provide a substantial improvement over current thresholding schemes. One advantage of the error model is that the system parameters inherently specify the properties of both the additive and multiplicative error terms. The method of the invention further provides for the performance of a generalized likelihood ratio test for each analyte to determine whether the amounts are relatively different.

In one embodiment, the method of the invention provides a refined test for comparison of differentially expressed genes that does not rely on gene expression ratios, but directly compares a series of repeated measurements of two observed intensities for each gene. In this regard, the method of the invention utilizes an error model and an associated significance test to determine whether the observed amounts of genes are significantly different between the two or more conditions being compared.

As used herein, the term “analyte” refers to a molecule whose presence is measured. An analyte molecule can be essentially any molecule for which a detectable probe or assay exists or can be produced by one skilled in the art. For example, an analyte can be a macromolecule such as a nucleic acid, polypeptide or carbohydrate, or a small organic compound. Measurement can be quantitative or qualitative. An analyte can be part of a sample that contains other components or can be the sole or major component of the sample. Therefore, an analyte can be a component of a whole cell or tissue, a cell or tissue extract, a fractionated lysate thereof or a substantially purified molecule. Moreover, an analyte can incorporate a second molecule, for example, a detectable moiety such as a dye, radiolabel, heavy atom label, or other mass label, a fluorochrome, a ferromagnetic substance, a luminescent tag or a detectable binding agent such as biotin. The analyte can be attached in solution or solid-phase, including, for example, to a solid surface such as a chip, microarray or bead.

As used herein, the term “sample” refers to the substance containing the analyte. It can be heterogeneous or homogeneous. Examples of heterogeneous samples include tissues, cells, lysates and fractionated portions thereof. Homogeneous samples include, for example, isolated populations of polypeptides, nucleic acids or carbohydrates. A sample can also be a purified analyte, free from like or non-like molecules. All of such substances are included within the meaning of the term so long as the substance contains the analyte. In addition to containing the analyte, a sample further can contain one or more additional components such as a buffer, detectable moiety, nucleic acids, polypeptides, carbohydrates or any other substance or molecule.

As used herein, the term “signal” is intended to mean a detectable, physical quantity or impulse by which information on the presence of an analyte can be determined. Therefore, a signal is the read-out or measurable component of detection. A signal includes, for example, fluorescence, luminescence, calorimetric, density, image, sound, voltage, current, magnetic field and mass. Therefore, the term “observed signal,” as used herein is intended to mean the actual quantity detected of the measured analyte in a particular detection system. An observed signal can include subtraction of non-specific noise. An observed signal can also include, for example, treatment of the measured quantity by routine data analysis and statistical procedures which allow meaningful comparison and analysis of the observed values. Such procedures include, for example, normalization for direct comparison of values having different scales, and filtering for removal of aberrant or artifactual values. A “mean signal” as used herein, refers to the true or inherent quantity of the measured analyte. A mean signal therefore corresponds to the detectable quantity of the analyte independent of variation in the assay or detection system.

As used herein, the term “sample pairs” refers to two samples containing analytes to be compared. The analytes to be compared within the two samples can be different, or they can be substantially the same species of analyte but subjected to distinct conditions or obtained from distinct sources. Therefore, the term “mean signal pairs,” as used herein, refers to the two true signals, one per analyte, associated with a sample pair. Similarly, when more than two analytes are being compared, the terms “sample sets” and “mean signal sets” are intended by analogy to reference the multiple samples containing the analytes and the corresponding multiple true signals, respectively.

As used herein, the term “system parameter” refers to the properties of the noise of the system, such as non-analyte, non-specific background signals. Therefore, the system parameter, designated β, is a measure of the error of the system and corresponds to undesirable or interfering signals that distort the true signal.

As used herein, the term “significantly unequal” refers to two analytes that have a meaningful difference in signal. Therefore, significantly unequal signals refers to two or more signals whose difference is caused by something other than chance, including variation or error in the system.

The invention provides a method of determining a true signal of an analyte. The method consists of measuring an observed signal x for one or more analytes and determining a mean signal (μ) and a system parameter (β) for the analyte that produce enhanced values for the probability likelihood of the observed signal, which is related to the mean signal by an additive error (δ) and a multiplicative error (ε), where the system parameter specifies the properties of the additive error (δ) and of the multiplicative error (ε).

The invention further provides a method determining relative amounts of an analyte between samples. The method consists of measuring observed signals x and y for an analyte within two or more sample pairs, determining a mean signal pair per analyte (μ) and a system parameter (β) for each sample pair, that produce enhanced values for the probability likelihood of the observed signals, which are related to the mean signal by an additive error (δ) and a multiplicative error (ε), where the system parameter specifies the properties of the additive error (δ) and the multiplicative error (ε).

The methods of the invention permit determination of the mean signal, which is the true amount of an analyte, by taking into account both multiplicative and additive error contributions to each observed signal. The methods of the invention further allow accurate determination of relative amounts of an analyte between samples. A maximum-likelihood approach is used to fit the model to observed signals of the analyte. The method of the invention can be used to monitor error introduced by intrinsic or extrinsic factors, to monitor total amount of error over time as well as to isolate or identify particular samples that have a higher error than normally observed. Therefore, the methods of the invention can be used to detect error introduced during any step in the analyte preparation and measurement. Additionally, the methods of the invention can be used, for example, to detect total error of the system or to separate and dissect biological or other intrinsic sample error from assay and procedure error. Thus, the methods of the invention allow quantitative analysis of the mean or true amount of an analyte at any given end point in a procedure as well as allow dissection of the system or procedure to quantitatively determine either or both intrinsic or extrinsic error introduced at any given step of the procedure.

Likelihood methods use statistical data and probability models to provide optimal use of statistical information. Because likelihood methods provide a specific description of the pattern of variation in data, these methods can be used for estimation and hypothesis testing, which is a formal process of using data to make statistically meaningful decisions such as whether relative amounts of analyte are significantly different between samples. Therefore, the methods of the invention determine, by formal estimation procedures, the mean signal of an analyte or a comparison of mean signals to provide the relative levels of the corresponding analyte. The comparison of mean signals can be for the same analyte subjected to two or more different conditions, different analytes under the same conditions or any combination thereof.

For comparison of two signals, the maximum-likelihood approach provided by the invention has several advantages over currently accepted ratio-based significance tests. In the ratio-based method, the expression ratio for the two signals to be compared is computed and compared to a control or reference ratio. For example, where the relative level of an analyte is to be compared under two different conditions, the ratio rᵢ=xᵢ/yᵢ is computed for analyte i for the two conditions x and y, and compared to a reference ratio of known analyte signals. A ratio that differs from the reference ratio, for example, as rᵢ>r_{c} or rᵢ<1/r_{c} identifies the analyte levels under the two conditions as being meaningfully different. This ratio-based method has been widely used in fields that compare, for example, the differences in expression of RNA or protein under two different conditions. The method has been particularly applicable to large scale expression analysis such as those utilizing microarray formats. However, the ratio-based method for statistical analysis of signal data combines observed signals into a single ratio, which necessarily results in the loss of absolute signal information. Moreover, when repeated samples per analyte are available, common practice is to compute the ratio of averaged signals, again discarding useful information.

The methods of the invention are generally applicable to measure any analyte that serves as a sample or is contained in a sample so as to allow for detection of the presence of the analyte. As will be described in further detail below, detection of the analyte signal can be by any means as long the observed signal allows for determination of a mean.

Once a signal indicating the presence of an analyte has been observed, the methods of the invention can be used to determine the true or mean signal of the analyte. The true signal of an analyte is independent of experimental variation or error introduced prior to or during detection of the observed signal. Removal of such error in a signal allows for more accurate quantitation of an analyte and reproducibility of measurements. Therefore, the true or mean signal of an analyte is a measurement of the true or actual level of that analyte. Moreover, through determination of the true signal, the methods of the invention can measure the reproducibility of steps in a process such as, for example, manipulations prior to the determination of the observed signal.

The methods of the invention are applicable to the measurement of analytes and determination of true signals in both biological and non-biological settings. For example, in a biological setting, experimental error can be classified into at least two categories. Biological error is one such category and consists, for example, of intrinsic error introduced by the biological components. In this regard, regulation at both the gene expression and protein activity levels can be substantially altered due to apparent negligible experimental differences in the treatment of a biological sample. A specific example is where gene expression changes due to the use of different batches of the same media during the course of an experiment. Such biological error produces measurable differences in the level of an analyte such as an expressed gene.

Another category is the extrinsic error introduced through experimental manipulation. For example, differences in sample preparation, analyte or probe labeling efficiency, hybridization or binding conditions, synthesis of probes, batches of solid-phase substrate and detection efficiency introduce variations in the determination of a measured analyte, even though all components and processes can be controlled so as to result in apparent negligible differences. Nevertheless, measurable differences in observed analyte signal occur due to the introduction of such error.

Similarly, for non-biological settings the methods of the invention are applicable for determination of true signals from measured analytes in essentially any process or steps thereof for which a quantitative determination or comparison of a measurable component is desired.

The above exemplary, and other forms of error all affect the perceived amount of a measured analyte through the introduction of fluctuations in the observed signal. Assessing the true signal of the analyte, independent of such fluctuations, allows direct comparison of analyte levels. Moreover, because the true signal of an analyte measurement can be determined, the methods of the invention provide a means for a direct or standardized comparison of analyte measurements both within an experimental system and between different systems. Given the teachings and guidance provided herein, essentially any analysis format known in the art can be used for such subsequent comparison of analytes once the true or mean signals are obtained. Therefore, the methods of the invention can be used to accurately and reproducibility determine the true signal of essentially any measurable analyte as well as used for the initial step in, for example, a comparative analysis of the same analyte under different conditions, the same analyte under repetitive conditions or different analytes under the same conditions.

As will be described further below, it is understood that the methods of the invention are equally applicable to both large and small sets of analyte samples and sets of measurements. Determination of the true signal for an individual sample is performed similarly as that for the determination of many, and even hundreds or thousands of samples. Similarly, the comparison of true signals for determination of relative amounts of an analyte between samples also is performed for two samples as it is for comparison of many sample pairs or higher order sets of multiple comparisons. Therefore, given the teachings and guidance provided herein, the number of true signals that can be simultaneous determined, or sets of samples that can be simultaneously compared for relative amounts of true signal is only limited by the available computational power.

The methods of the invention for determining the true signal of an analyte can be applied to a variety of situations. For example, repeated measurements of the observed signal such as intensity x for one or more analytes can be obtained and subsequently used in the method of the invention to characterize the error and determine the significance value for each observed signal. For example, repeated observations of the signal associated with a single analyte such as, for example, the observed intensity of a single gene in a microarray, can be utilized in the methods of the invention to monitor, for example, the variation introduced by two or more distinct conditions, the total error introduced over a given time or sporadic error introduced by any means including variation caused at any step in the protocol.

The method of the invention provides a description of the relationship between an observed signal and a mean signal. The relationship specifies that the observed signal can be described as containing both an additive error term and a multiplicative error term. The error terms are a measure of variation in the observed signal. Parameters of the additive error term and the multiplicative error term set forth the characteristics or features of the error terms. These parameters are derived from statistical relationships well known in the art. Therefore, the error terms, and the parameters defining them, specify the noise of the analyzed system. Knowing the components and relationship of the noise with reference to the mean or true signal allows determination of the true signal given an empirically measured signal.

The inclusion of both an additive error term and a multiplicative error term in the described relationship permits distinction of the true signal from the noise at a wide range of observed signals. For example, with a high observed signal, or high observed signal relative to the noise, the system noise can be primarily described by the multiplicative error term. Therefore, the true signal can be accurately distinguished from the noise by employing only a multiplicative error term in the method of the invention. In contrast, where the observed signal is low, or low relative to the noise, the influence of the additive error in describing the noise becomes substantially more prominent. Maintaining this error term in the described relationship at low observed signals enhances the accuracy in distinguishing the true signal from the noise. Similarly, at intermediate observed signal ranges, both the additive and multiplicative error terms substantially influence the description of the noise and inclusion of both will yield enhanced results in distinguishing the true signal from the noise using the described relationship in the method of the invention. Therefore, including both the additive and multiplicative error terms in the description of the relationship between the observed signal and the true signal results in more accurate and predictable performance of the method of the invention at all ranges of observed signal.

However, utilization of both the additive and multiplicative error terms in the methods of the invention is not always necessary. As described above, if the user knows or can determine that the observed signal is high relative to the limits of detection or relative to the noise, then determination of the true signal can be accurately made by inclusion of only the multiplicative error term. In such circumstances, the additive variation will be small or negligible compared to the observed signal and is included in the described relationship as an example where one or more of the error term parameters, such as the standard deviation of the additive error term, is set to zero. Similarly, where the signal is low but the variation is also known, or can be determined to be small, in like manner the additive error term also can be omitted without substantial affect on determination of the true signal. Determination of the true signal also can be accurately made by inclusion of only the additive error term. For example, applying only the additive error term in the described relationship can be useful for measuring the error in the variation of the background of a system. Given the teachings and guidance described herein, those skilled in the art will know, or can determine, whether determination of a true signal can be made, or is desirable, utilizing both the additive and multiplicative terms in the described relationship employed in the method of the invention.

For each analyte, the method of the invention provides a relationship between the observed signal and the mean signal which can be described as follows:

xᵢⱼ=μₓᵢ+μₓᵢεₓᵢⱼ+δₓᵢⱼ,

where each measurement j equals 1 through M and each analyte i equals 1 through N, and where xᵢⱼ is the observed signal and μₓᵢ is the mean or true signal. For each analyte and measurement, the multiplicative error term, εₓ, and the additive error term, δₓ, can be obtained, for example, from a normal distribution with mean zero and standard deviation σ_{εx} and σ_{δx}, respectively. One advantage of the above described relationship is that the multiplicative and additive errors can be independent of one another. Additionally, the additive and multiplicative error terms can be derived from a variety of univariate distributions, including, for example, a parametric distribution, a univariate normal distribution, a t-distribution or a gamma distribution.

For determining the true signal of an analyte, where the observed signal xᵢⱼ is described by a univariate distribution with the parameters μₓᵢ and σₓᵢ, the error model specifies two analyte-independent parameters, which together consist of the system parameter (β), and a mean signal βₓ for the analyte. The system parameter β describes the noise in the observed signal and consists of the above described standard deviation of the multiplicative error with respect to the mean (σ_{δx}) and the standard deviation of the additive error with respect to the mean (σ_{δx}) A particular feature of the above relationship, or error model, is that it specifies both the mean signal and noise such that the estimate of the signal describes the structural features of the noise. Therefore, the system parameter specifies the properties of both the multiplicative and additive error and can be independent of the mean signal. Moreover, the error terms specified in the model can be independent of one another.

Modifications can be incorporated into the general description of the relationship between the observed signal and the mean signal set forth above and below which do not alter the relationship of the additive or multiplicative error terms with respect to the true signal or their properties in specifying the structure of the noise. Such modifications are exemplified with reference to the description specifying the relationship between the observed signal and the true signal set forth above, but are similarly applicable to the description specifying the relationship between observed and true signals for comparison of two or more signals. The modifications can include, for example, inclusion of functions, augmentations or addition of terms, simplification or removal of terms and transformation of variables. Depending on the origin of the signal data or the desired use, one or more of such modifications can be employed to generate alternative forms of the described relationship appropriate for application to a wide variety of data sets. These modifications as well as others are well known to those skilled in the art and are applicable in the method of the invention.

For example, the description specifying the relationship between the observed and true signal can be modified by inclusion of a function such as f(σₓᵢ)εₓᵢⱼ where f is a function that describes how the mean sensitive component of variability varies as the mean varies. The function can do so simply by multiplying the mean signal by δₓᵢⱼ, or it can do so by multiplying εₓᵢⱼ by other terms related to the mean, in addition to the mean or together with the mean. Additionally, the system parameters also could be chosen as a function of the mean parameter μₓᵢ. For example, and with respect to the expanded relationship set forth below describing the comparison of two or more true signals, the system parameters ρ_{ε} and ρ_{δ} can be chosen as a function of the mean parameters μₓᵢ and μ_{yi}. With either of the above exemplarily functions, the system parameters would change according to principles well know to those skilled in the art to reflect the joint properties of the error of the system given the teachings and guidance provided herein. For example, the function “f” can be chosen to be a polynomial of low order and the system parameter would be enlarged to include the coefficients of these enlarged polynomials.

The description specifying the relationship between the observed and true signal also can be modified by augmentation. For example, terms can be added which include constants, second order or even higher order terms which do not alter the relationship of the additive or multiplicative error terms with respect to the true signal or their properties in specifying the structure of the noise. A specific example of the addition of a constant is xᵢⱼ=μₓᵢ+μₓᵢεₓᵢⱼ+δₓᵢⱼ+C, where C is a global parameter which allows, for example, translation of the relationship along selected axes. Shifting the distribution by a constant can be useful, for example, in the normalization process to better fit the data as a whole. Additionally, a specific example of the addition of a second order term is xᵢⱼ=μₓᵢ+(μₓᵢ+αμ²ₓᵢ)εₓᵢⱼ+δₓᵢⱼ. A specific example of the addition of a higher order term is xᵢⱼ=μₓᵢ+(μₓᵢ+αμ²ₓᵢ+βμ³ₓᵢ)εₓᵢⱼ+δₓᵢⱼ. These latter two descriptions allow for curvature in the relationship between the mean signal and the standard deviation at medium-to-large signal intensities.

Simplification or removal of terms has been described above, such as when there is a negligible amount of error. Removal of the corresponding error term can increase the accuracy of determining the remaining parameters and therefore the accuracy of determining the true signal. A specific example of a simplification modification where the additive error has been removed is xᵢⱼ=μₓᵢ+μₓᵢεₓᵢⱼ.

Transformation of variables is yet another modification which can be performed that does not alter the relationship of the additive or multiplicative error terms with respect to the true signal or their properties in specifying the structure of the noise. For example, because some signal measurements can be distributed over a large range of values, including many orders of magnitude, it can be useful to transform the raw signal measurements into logarithms. For this transformation, the variables xᵢⱼ, or for example yᵢⱼ in the relationship set forth below, can be redefined in terms of other variables such as s and t. Specifically, define s log(xᵢⱼ) and take the log of both sides of the equation: log(xᵢⱼ)=log(xᵢⱼ=μₓᵢ+μₓᵢεₓᵢⱼ+δₓᵢⱼ). In the specific case where the additive error is small, the above equation reduces to: log(xᵢⱼ)=log(μₓᵢ)+log(1+εₓᵢⱼ). Substituting s=log(xᵢⱼ), this equation relates the sample value of s to the mean of s plus some additive error f=log(1+εₓᵢⱼ), as in: s=μₛ+f. Other transformations include, for example, exponentiation (s=εⁿₓᵢⱼ) or polynomial transformations (s=axₙᵢⱼ).

The methods of the invention employ the above error model to determine, by formal estimation, the mean signal of an analyte from a set of measurements of an observed signal by using a maximum likelihood approach. To estimate the mean signal, the observed signal should be measured at least twice (j=2), obtaining two separate values and allowing for a more accurate computation of the system parameter and mean signal. However, a larger number of analyte measurements, where j is greater than 2, results in further refinements of true signal determination. For example, as shown in Example I, increasing the number of measurements from two to four per analyte results in beneficial enhancements in true signal determination. Therefore, the number of measurements of a particular analyte can be a few or many times, including for example, about 2, 3, 4, 5, 10, 20, 50, 100 or more sample measurements. Although as few as two measurements is sufficient to accurately determine the true signal of an analyte, the actual number of measurements will vary depending on the need and confidence requirement of the user. For example, the confidence in true signal determination can be increased in analyte samples exhibiting inherently greater variation by compensating for the greater experimental error through increasing the number of sample measurements. Sample measurements can be derived, for example, from independent samples, replicates of the same sample that are independently measured, repeated measurements of the same sample or any combination thereof.

Once the signal has been measured for one or more analytes, the observed signals can be subjected to a variety of statistical methods well known in the art to prepare the raw data for maximum likelihood analysis. Such methods include, for example, standardization and filtering techniques. Briefly, non-specific background can be subtracted to produce, for example, the observed signal x′. Moreover, depending on the need, the data measurements can be, for example, normalized to have comparable medians and extreme signals within a set of multiple measurements that are artifactually outside the signal range of its partners can be removed. Such modified values for the observed signal are similarly applicable in the methods of the invention for determining the true signal of an analyte. Therefore, the error model of the invention additionally accounts for the influence of multiplicative and additive errors on the observed signals and provides a relationship between an observed signal x′, and the corresponding mean or true signal.

As will be described further below in context of a comparing relative differences between two or more true signals, once obtained for any particular set of analyte measurements, the observed signal x or x′ is analyzed by, for example, maximum likelihood probability for determination of its mean signal. In addition to a maximum likelihood approach, other approaches are known in the art to determine, by formal estimation, the mean signal from a set of observed measurements, including, for example, Quasi-Maximum Likelihood and Generalized Method of Moments.

In addition to determining the true signal of an analyte, the methods of the invention also can be utilized to determine relative amounts of an analyte between samples. Briefly, following the methods described above for determination of a true signal for an individual analyte, for comparison of relative amounts of two or more analytes, observed signals are measured for each analyte and the corresponding true signals determined by probability likelihood analysis. The resultant true signals are then formally assessed by, for example, a difference indicator to determine relative levels. In this embodiment, for example, the methods of the invention identify true signals that are significantly unequal, thus representing different amounts of analytes between the compared samples.

The methods of the invention allow relative comparison of true signals between two analytes or pairs as well as between multiple analytes or sets. As described previously, the analytes to be compared can be can be different, or they can be substantially the same species of analyte but subjected to distinct conditions or obtained from distinct sources. Briefly, samples harboring analytes to be compared are referred to herein as sample pairs or sets. True signals resulting from each observed analyte signal for a particular comparison are similarly referred to as mean signal pairs or mean signal sets. Similarly, the true signals being compared for substantially the same analyte species derived from different conditions or sources is referred to herein as mean signal pairs per analyte and mean signal sets per analyte.

By reference to comparison of two analytes, for the determination of relative amounts of an analyte between samples the observed signal and mean signal within a sample pair can be described by the following relationship:

xᵢⱼ=μₓᵢ+μₓᵢεₓᵢⱼ+δₓᵢⱼ, and

yᵢⱼ=μ_{yi}+μ_{yi}ε_{yij}+δ_{yij}

where each measurement j equals 1 through M and each analyte i equals 1 through N; where xᵢⱼ and yᵢⱼ are the observed signals, and where μₓᵢ and μ_{yi} are the mean signals. For each pair of analytes and measurements, the multiplicative error terms, εₓᵢⱼ and ε_{yij}, can be obtained, for example, from a bivariate normal distribution with mean zero and standard deviations σ_{εx} and σ_{εy}, and correlation ρ_{ε}. Similarly, the additive error terms, δₓᵢⱼ and δ_{yij} also are drawn from a bivariate normal distribution with mean zero and standard deviations σ_{δx} and σ_{δy}, and correlation σ_{δ}. Aside from the correlations already described, the error terms for a particular analyte i can be independent, that is, the multiplicative error terms (εₓᵢ and ε_{yi}) can be independent of the additive error terms (δₓᵢ and δ_{yi}) and the error terms for analyte i (εₓᵢ, ε_{yi}, δₓᵢ, δ_{yi}) can be independent of the error terms for analyte j (εₓⱼ, ε_{yj}, δₓⱼ, δ_{yj}) when j does not equal i (j≈i). Additionally, the additive and multiplicative error terms can be derived from a variety of other bivariate distributions, including for example, a parametric distribution, a bivariate normal distribution, a t-distribution or a gamma-distribution, and, further, the independence assumptions can be dropped by including additional correlations in the system parameter β.

The above described relationship between observed and mean signals for two analytes substantially parallels that described previously for an individual analyte. Therefore, this error model similarly provides the advantage of allowing multiplicative and additive errors to be independent of one another. Similarly, the above described error model can be applied by analogy to determination true signals for multiple analytes, including three or more analytes. For example, similar mean signal, multiplicative and additive error terms for analyte z can be described in a third equation. Additionally, higher order comparisons and error models can additionally be described using the teachings and guidance provided herein.

For determining the true signal of an analyte pair, where, for example, the observed signals xij and y are described by a bivariate distribution with the parameters μₓᵢ, μ_{yi}, σₓᵢ, σ_{yi} and ρ_{xiyi} the error model specifies six analyte-independent parameters, which together consist of the system parameter β=(σ_{εx}, σ_{εy}, ρ_{ε}, σ_{δx}, σ_{δy}, ρ_{δ}), and a mean signal pair, (μₓᵢ, μ_{yi}) for the analyte. As with the univariate distribution described previously, the system parameter β for the bivariate distribution similarly describes the noise in the observed signal and consists of the above described standard deviation and correlations. Briefly, the analyte-independent parameters of the system include the standard deviation of the multiplicative error with respect to the mean of signal x (σ_{εx}), the standard deviation of the multiplicative error with respect to the mean of signal y (σ_{εy}), a correlation of the multiplicative error for the mean of signals x and y (ρ_{ε}), the standard deviation of the additive error with respect to the mean of signal x (σ_{δx}), the standard deviation of the additive error with respect to the mean of signal y (σ_{δy}) and a correlation of the additive error for the mean of signals x and y (ρ_{δ}). As described previously, one particular feature of the above relationship, and with the error models of the invention, is that it specifies both the mean signal and noise such that the estimate of the signal describes the structural features of the noise. Therefore, the system parameter specifies the properties of both the multiplicative and additive error and can be independent of the mean signal. Moreover, the error terms specified in the model can be independent of one another.

To determine, by formal estimation, the mean signal pairs of a sample pair, the observed signals x and y should be measured at least twice as described previously. Once the signals have been measured for analytes within one or more sample pairs, the raw data can be prepared for maximum likelihood analysis to produce, for example, two signals x′ and y′. For analysis of more than two analytes within a sample pair, standardization and filtering methods can similarly be used to produce, for example, signals z′ and the like for sample sets. These methods and others well known in the art for processing raw data into useful statistical form are particularly appropriate when analyzing multiple observed signals of sample pairs and sets in order to provide meaningful comparisons by, for example, normalization of divergent scales for the initially measured signals. Such modified values for the observed signals are similarly applicable in the methods of the invention for determining mean signal pairs, mean signal pairs of an analyte and mean signal sets. Therefore, the error model of the invention additionally accounts for the influence of multiplicative and additive errors on the observed signals and provides a relationship between observed signals x′, y′, z′ and higher numbers of like comparisons, and the corresponding true signals.

For any of the error models described above, once an observed signal, observed signals within a sample pair or sample set are obtained, the mean signal (μ) and the system parameter (β) can be determined or selected by, for example, a non-linear optimization algorithm. Such statistical optimization procedures are well known in the art and can be applied to, for example, individual observed analyte signals, observed signals for a single sample pair and to observed signals for two or more, including, for example, hundreds, thousands or ten thousand or more signals for sample pairs or sets. The number of optimizations that can be performed is coextensive with the number of analyte signals or higher order sets that can be measured and the computing power available in the art.

Similarly, and in addition to non-linear optimization algorithms, any general optimization procedure for non-linear equations can be used to determine or select the mean signal pair (μ) and a system parameter (β) for each sample pair including, for example, Gradient Descent, Newton-Raphson and Simulated Annealing. For example, The Gradient Descent method is based upon selecting, at each iterative step, the direction in multidimensional space for which the objective function initially changes at the fastest rate, and subsequently choosing an appropriate distance to move in this direction at that iterative step. The Newton-Raphson method is based on a linear approximation to the first-order conditions, which may be numerically estimated, that set to zero the partial derivatives of the objective function with respect to the parameters being estimated. The Simulated Annealing method is based upon making random changes, which become smaller throughout the iterations, in the parameters being estimated and subsequently deciding probalistically whether or not to keep these changes, thereby seeking an optimum while maintaining the ability to escape from a suboptimal local optimum in order to seek a better solution.

Further, the methods of the invention also allow the mean signal and system parameter to be provided based on previously determined or estimated values rather than calculated de novo. For example, in routine or familiar procedures, the user can have prior knowledge of beneficial or optimal estimates that can be used to calculate enhanced values for the probability likelihood or which more efficient convergence to a maximum probability likelihood. Therefore, the mean signal pair, including the mean signal pair per analyte, for example, (μ) and a system parameter (β) for each sample pair can be determined or provided and then subsequently compared. As will be described further below, comparison of mean signals, mean signal pairs and higher order sets can be performed, for example, by identification of significantly unequal mean signals using well known methods in the art such as statistical difference indicators.

In one embodiment, the mean signal and system parameters are estimated using maximum likelihood estimation. The maximum likelihood function provides, for example, a framework for the formal estimation process, while recognizing the structure of the random noise in the system. By modeling patterns of randomness, the maximum likelihood estimation process can better separate and estimate the signal. The method of the invention provides likelihood functions using estimates for the true parameters by utilizing standard optimization procedures as described herein. One advantage of the methods of the invention is that, if desired, the error terms can be independent of one another. Moreover, each mean signal within a mean signal pair or set also can be independent with respect to each other. These characteristics allow for the independent optimization of the system parameter and mean signal. Therefore, the efficiency of optimization can be significantly increased for a large number of analytes, for example, through the optimization of the system parameter and mean signals in subsets.

Briefly, observed values are measured and, subsequently, the system parameter (β) can be selected to enhance the probability likelihood given the observed signal. Similarly, for each analyte, mean signal pairs can be selected to enhance the probability likelihood given the system parameter (β). The mean signal pair and system parameter can be determined at the same time, or alternatively, the mean signal can be determined prior to the system parameter and then subsequently used to determine the system parameter. Conversely, the system parameter can be determined prior to the mean signal and then subsequently used to determine the mean signal. As described further in Example I, this procedure can be reiterated one or more times until the mean signal pair per analyte (μ) and a system parameter (β) converge. With each selection of values and reiteration of the optimization procedure, the calculated mean is enhanced in the direction of the true signal for that analyte, pair or set. In addition to maximum likelihood estimation, probability likelihood values for system parameters and mean signal can be estimated using other modeling techniques known in the art including, for example, Quasi-Maximum Likelihood and Generalized Method of Moments.

For comparison of the relative levels of two or more true signals, after the system parameter and mean signal have been determined, the methods of the invention provide for identification of mean signal pairs that are significantly unequal, representing different amounts of analytes between the compared samples. The error models and methods of the invention take into account the observation that x and y variances and x-y correlation increase with increasing values of x and y. Based on these empirical observations, the methods of the invention utilize a likelihood ratio test to identify analytes whose true signals μₓ and μ_{y} are unequal. For example, in the case of RNA expression analysis, analytes with unequal mean signals have different copy numbers of the measured mRNA analyte in the two cell populations under comparison, or in other words, are differentially-expressed. Such methods for assessing significantly unequal mean signals are well known in the art and are described further below in the Examples. Thus, the methods of the invention provide a difference indicator for comparison of true signals and therefore relative amounts of two or more analytes. Additionally, when used in combination with known analyte standards, the methods of the invention can be employed to quantitate the amount of a test analyte by comparison of its true signal with that of one or more known standards.

The methods of the invention can be utilized for determining the true signal of an analyte or for comparing the relative levels of two or more true analyte signals in a variety of different formats and modified procedures. For example, observed signals for one or more analytes, sample pairs or sample sets can be measured independently, such as in series, or simultaneously, such as in parallel. Moreover, different observed signals can be measured, for example, from independent samples, the same sample or from independent samples that have been pooled to reduce the total number of samples which are to be manipulated. The number of different observed signals which can be measured from a single sample or pooled sample will depend, for example, on the number of unique detection labels which can be employed to uniquely measure each different analyte within the sample. Corresponding mean signals, mean pairs or mean sets can similarly be determined from the observed signals in series or parallel, for example. Additionally, the measurements of observed signals and determination of mean signals can be multiplexed with ongoing measurements and determinations proceeding simultaneously in series or parallel, such as in an automated system, for example.

Various modification can be made to the procedure described above for determining or comparing true signals which enhance the description of the noise and therefore, further increase the accuracy of distinguishing the true signal from the noise. For example, variation of a reference signal can be captured or incorporated into the analysis. In this specific example, two or more observed signals to be compared are first independently compared to a reference signal to determine, for example, the system parameters or mean signal pairs for each test-reference comparison. A probability likelihood can then be generated from the product of the terms for each initial test-reference comparison, to describe, for example, β₁ and β₂. These system parameters obtained with respect to the test-reference comparison can then be used, for example, to determine the mean signal pairs or sets for the two or more observed signals to be compared. Briefly, and as described further below, a likelihood is then established as the product of Lᵢ(β₁, μ¹ₓᵢ, μ¹_{yi}) and Lᵢ(β₂, μ²ₓᵢ, μ²_{yi}). A statistical difference indicator can then be applied, for example, constraining μ¹ₓᵢ and μ¹⁼²ₓᵢ, as well as μ¹_{yi} and μ²_{yi} to be equal or not equal to each other as described previously. For the specific example where y represents the reference sample, then μ¹_{yi} and μ²_{yi} would be constrained to be equal. Variation can be captured from one or more reference signals alone or in combination. Additionally, using the teachings and guidance provided herein, other methods well known to those skilled in the art which enhance the description of the signal or noise can additionally be incorporated into, or used in conjunction with the methods of the invention.

Therefore, the invention provides a method of determining relative amounts of an analyte between samples. The method consists of: (a) obtaining a reference signal; (b) obtaining observed signals x and y for an analyte within two or more sample pairs; (c) determining system parameters (β₁, β₂) for a sample pair comprising said observed signals x or y and said reference signal that provide a probability likelihood of said occurrence given said observed and reference signals, said observed and reference signals being related to said mean signal by an additive error (δ) and a multiplicative error (ε), where said system parameter specifies the properties of said additive error and said multiplicative error; (d) determining mean signal pairs (μ₁, μ₂) for said sample pair comprising maximizing a product of terms for said probability likelihood of said sample pair of observed signals x or y and said reference signal for said analyte, and (e) selecting a mean signal μₓ or μ_{y} that provides a maximum probability likelihood of occurrence given said observed signals and system parameters β₁ and β₂.

The invention also provides a method of determining relative amounts of large numbers of analytes between samples. The method consists of: (a) obtaining observed signals x and y for a plurality of immobilized analytes within two or more sample pairs; (b) determining a mean signal pair per analyte (μ) and a system parameter (β) for each sample pair that provides a maximum probability likelihood of occurrence given the observed signals, the observed signals being related to the mean signal by an additive error (δ) and a multiplicative error (ε), where the system parameter specifies the properties of the additive error and the multiplicative error, and (c) identifying one or more mean signal pairs per analyte that is significantly unequal. The method is applicable, for example, to nucleic acid and polypeptide analytes using immobolized array formats.

The methods of the invention are applicable for determination or comparison of true signals in a wide variety of systems. Various detection methods for numerous analytes are well known to those skilled in the art. All that is needed to practice the methods of the invention are measurable quantities of an analyte in a data form that can be calculated as a mean.

In biological systems, for example, detection of a nucleic acid analyte can be by any of a variety of detection methods well known to those skilled in the art. Such methods include, for example, gels, blots, capillaries and microarray formats. In addition to nucleic acid microarrays or chips, the methods of the invention further can be applied to determine the true signal of polypeptide spotted on a chip. The construction of glass chips or other substrates spotted either with chemicals to bind polypeptides or with known antibodies can be constructed and the bound polypeptide analyte can be detected, for example, by a mass spectrometer. Moreover, detection of a polypeptide analyte also can be by any other of a variety of detection methods well known in the art, including, for example, gels, blots, capillary and FACS formats. In addition, analytes other than nucleic acids and polypeptides can be detected by methods known in the art such as spectroscopy and laser-assisted techniques. The detection method and, consequently, the visualization technique that yields the observed signal will depend on a variety of factors such as the nature, amount, stability and purity of the analyte.

Microarray hybridization and fluorescent detection is one well known method for analysis of large numbers of nucleic acid analytes. Currently, arrays with more than 250,000 different oligonucleotide probes or 10,000 different cDNAs per square centimeter can be produce in significant numbers. Although it is possible to synthesize or deposit DNA fragments of unknown sequence, generally, microarray-based formats utilize specific sequences attached to a solid substrate such as glass, plastic, silicon, gold, a gel or membrane, beads, or beads at the ends of fibre-optic bundles. Such formats allow for parallel hybridization and simultaneous detection of a large number of indexed, surface-bound nucleic acid probes.

Nucleic acid arrays are generally produced by either robotic deposition of nucleic acids such as PCR products, plasmids or oligonucleotides, onto a glass slide or in situ synthesis using, for example, photolithography of oligonucleotides. After hybridization of labelled samples to the spotted or synthesized probes, the arrays are scanned and a quantitative fluorescence image along with the known identity of the probes is used to detect the presence of a particular molecule above thresholds based on background and noise levels.

Various methods for preparing labelled material for measurements of gene expression microarrays are well known in the art. For example, the RNA can be labelled directly, using a psoralen-biotin derivative or by ligation to an RNA molecule carrying biotin, labelled nucleotides can be incorporated in cDNA during or after reverse transcription of polyadenylated RNA; or cDNA can be generated that carries a T7 promoter at its 5′ end. In the last case, the double-stranded cDNA serves as template for a reverse transcription reaction in which labelled nucleotides are incorporated into cRNA. Commonly used labels include the fluorophores fluorescein, Cy3 or Cy5, or nonfluorescent biotin, which is subsequently labelled by staining with a fluorescent streptavidin conjugate. Generally, cDNA from two different conditions is labelled with two different fluorescent dyes such as Cy3 and CyS, and the two samples are co-hybridized to an array. After washing, the array is scanned at two different wavelengths to detect the relative transcript abundance for each condition.

Another quantitation method which is useful for determining expression levels of polypeptide analytes is the isotope-coded affinity tag (ICAT) method (Gygi et al., Nature Biotechnol. 17:994-999 (1999)). Specifically, ICAT involves labeling two analyte samples differently by using stable isotopes, loading them into a mass spectrometer, and measuring the ratio of the two labels and thus the relative mass. ICAT can make any separation method, including HPLC and capillary electrophoresis, quantitative and, rather than using a ratio-based comparison, the methods of the invention can be applied to any of these separation methods to determine the true signal of a polypeptide analyte or the relative amounts of an analyte between samples.

Additionally, measurement of an analyte signal can be by a variety of other methods well known in the art, including, for example, light emission, radioisotopes, and color development. Briefly, detection can involve methods such as radioactive labeling of the analyte using metabolic labeling in an appropriate cell or in vitro labeling by RNA transcription or by coupled in vitro transcription-translation with appropriate radioactive amino acids. Additionally, covalent modification with a radioactive or fluorescent substrate using an appropriate enzyme or chemical modification can be employed. Moreover, an analyte can be covalently modified by incorporating a chemical moiety capable of being detected. For example, green fluorescent protein, Cy3, Cy5 and other fluorophores can be covalently attached to a polypeptide analyte. Similarly, biotin can be covalently attached to a polypeptide analyte and subsequently detected by streptavidin using detection methods known in the art. Other methods also can involve fusion of an appropriate detection molecule to the analyte. For example, the analyte can be fused to luciferase and detected by light emission or can be fused to lacZ and detected by appropriate calorimetric detection.

The methods of the invention have utility for a variety of applications. Although a standard microarray compares only two populations, a greater number can be cross-compared by hybridizing labeled probe, such as cDNA prepared from each cell population of interest, to that of a common reference population. The methods of the invention can thus be used to determine genes differentially-expressed between any two populations, even if they have not been directly involved together in a single hybridization experiment.

The error model of the invention does not distinguish between repeated samples drawn from multiple spots on a single array versus repeated samples drawn from multiple hybridizations to different arrays. Because multiple spots within an array show less variability and more dye-to-dye correlation than do multiple spots observed over several arrays, the error model of the invention can be applied to distinguish between these two types of sampling, resulting in a more sensitive or accurate likelihood ratio test. Systems which involve more than one level of sampling are well known in the art and can be addressed by utilizing a nested design model as described by Dunn and Clark, Applied Statistics: Analysis of Variance and Regression (John Wiley & Sons, Inc., New York, 1987), which is incorporated herein by reference.

The methods of the invention further can be utilized to place a confidence interval on the true signal difference between two analytes. In this embodiment, rather than testing the hypothesis that μₓ=μ_{y}, the range 1<(μₓ−μ_{y})<h or the range 1<(μₓ/μ_{y})<h is determined for each analyte.

In another embodiment, the methods of the invention can be utilized to quantify, compare, and ultimately reduce the error introduced by each stage of an array process. Therefore, the methods of the invention can be used for quality control in a large variety of processes and settings. For example, as shown in Example II, system parameters and mean signals can be compared for replicate spots on one array versus a single spot observed over multiple array hybridizations (see also Table 2). It is understood that this embodiment of the method of the invention can be expanded to quantify several different levels of variation, such as variation due to cell culture, RNA preparation, labeling, or hybridization. Moreover, it can be expanded to other biological assay systems as well as non-biological systems. Thus, the method of the invention can be utilized to identify sources of variation that contribute to the overall error of the system.

The methods of the invention can be extended to a wide range of biological data involving comparisons between multiple measurements and can be advantageously utilized to determine differential gene expression based on studies with fluorescent or radioactive-labeled cDNA hybridized to gene clones spotted on membranes. Furthermore, the methods of the invention are applicable to large scale genotyping of human polymorphisms, where normal DNA is cut into small fragments, labeled, transferred onto a microchip and subsequently hybridized with labeled samples of normal and polymorphic DNA. Because the observed quantities of polypeptide expression per gene are analogous to fluorescent signals observed in a microarray experiment and are correlated, the methods of the invention can be practiced with technologies for comparing levels of polypeptide expression between two cell populations, for example (Gygi et al., Mol. Cell Biol., 19:1720-1730 (1999), supra. Thus, the method of the invention can be advantageously utilized for describing measurements obtained in various technologies including those pertaining to, for example, genomics and proteomics.

For example, the method of the invention can be applied to proteomics where increased sensitivity of sequencing methods and mass spectrometry allow for determination polypeptide expression profiles. The methods of the invention can be advantageously used to determine relative amounts of polypeptide based on, for example, virtual 2-D profiles obtained by linking of isoelectric focusing gels with mass spectrometry.

It is understood that the observed signal depends on the method of detection. For example, in the case of a microarray, the amount of hybridization can be quantified by, for example, optical imaging or laser scanning to observe the emitted light intensity. The observed signal also can be obtained by other visualization techniques based on the nature of the analyte as well as the assay and include, for example, chemiluminescence and fluorescence imaging systems, and mass spectrometry. These and other methods are well known in the art and can be employed for the detection of an observed signal in the methods of the invention.

### EXAMPLE I

### Development of an Error Model of the Variability Observed Over Repeated Observations of Intensities for Genes Represented on a DNA Microarray

This example describes development of a maximum-likelihood test for the variability observed over repeated observations of intensities for genes represented on a DNA microarray.

Preprocessing of Microarray Data

The amount of hybridization to each spot is quantified by scanning the array with a laser and observing the intensity of light emitted. Observations are made separately for the two dyes, such that two intensities x and y are observed for each spot on the microarray. This process does not behave deterministically in practice, such that multiple spots corresponding to each gene i hybridized under identical conditions will result in a distribution of intensities xᵢⱼ and yᵢⱼ (1≦i≦N; 1≦j≦M), where N is the number of genes represented on the microarray and M is the number of spots observed for each gene.

Spot intensities were extracted from a scanned image, then background-subtracted and normalized as follows: microarray images are processed with Dapple, a software tool developed for array spot finding and quantitation described by Buhler et al., Bioinformatics 2000, which can be found at the URL: cs.washington.edu/homes/jbuhler/research/array, which is incorporated herein by reference. The Dapple software locates each spot and reports a separate median foreground intensity for each dye inside the spot area. The Dapple software also provides a local background intensity estimate for each spot and dye. The Dapple intensity estimates were subsequently smoothed by spatial filtering using a 7 spot by 7 spot median filter as described by Lim J. S. Two-Dimensional Signal and Image Processing (Englewood Cliffs, Prentice Hall, 1990), which is incorporated herein by reference. Subsequently, the smoothed background was subtracted from the foreground of each spot so as to produce the background-subtracted intensities x′ and y′.

In practice, X′ and y′ have different scales and thus are not directly comparable. This situation can occur if the total amount of labeled cDNA is greater for one dye than it is for the other, if one dye incorporates more efficiently, or if the scanner has different sensitivities to the two dyes. Therefore, the intensities are normalized to have identical medians A within each array hybridization:

$$x = \frac{{Ax}^{′}}{{\tilde{x}}^{′}} y = \frac{{Ay}^{′}}{{\tilde{y}}^{′}} A = \frac{1}{2} ( {\tilde{x}}^{′} + {\tilde{y}}^{′} )$$

where \tilde{X}′ denotes the median intensity of x′ over all spots on a single microarray. If multiple array hybridizations are performed, normalization occurs independently for each and the resulting combined data set consists of data pairs (xᵢⱼ, yᵢⱼ) for gene i in repeat j. If three or more samples are available for a gene, these are filtered independently in x and y to remove outliers by Dixon's test with a=0.l as described in Dunn and Clark, Applied Statistics: Analysis of Variance and Regression (2nd ed., Wiley and Sons, New York, New York, 1987), which is incorporated herein by reference. In addition, extremely high intensities outside the dynamic range of the array scanner in either color are removed.

Formulation of the Error Model

An error model summarizing the influence of multiplicative and additive errors on x and y has been formulated. In this regard, it has been consistently observed that larger intensity measurements have a proportionately larger error over repeated samples.

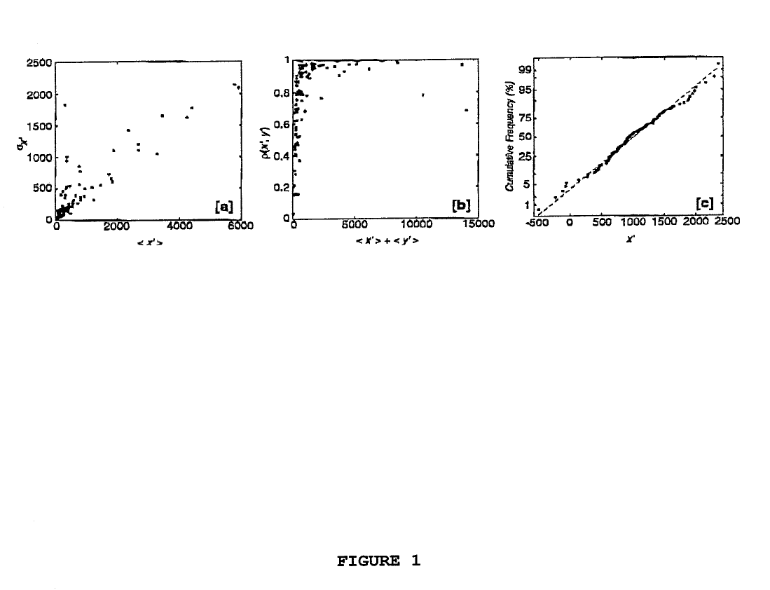

The data shown in FIG. 1, which shows the increase of (A) standard deviation and (B) correlation with absolute level of intensity x′ or y′, were obtained over 5 separate hybridizations with identically-prepared Cy3- and Cy5-labeled cDNA mixtures to test arrays containing 16 replicate spots per gene over 96 genes, resulting in a total of 80 samples for each of 96 genes. FIG. 1 (C) shows the normal probability plot for the 80 samples of x′ pertaining to a single, representative gene. This plot is linear, indicating that these data are consistent with a normal distribution. The dotted line connects the 25th and 75th percentiles of the data and represents an approximate linear fit.

As shown in FIG. 1(A), larger intensity measurements have a constant coefficient of variation σₓ∝x′, as can be caused by variation in spot size or labeling efficiency from gene to gene. However, the variability does not tend to zero as x_{→}0, likely due to variation in the measured background intensity. Furthermore, within genes, x and y are correlated and, in addition, larger intensities have a larger correlation, possibly due to errors introduced by spot-to-spot nonuniformity or during the hybridization process which affect intensity measurements for both dyes simultaneously (see FIG. 1B). Finally, as shown in FIG. 1B, samples of x and y for a given gene are at least approximately normally distributed, as assessed by a normal probability plot described by Dunn and Clark, supra, 1987.

Based on the observations described above, the background-subtracted, median-normalized intensities observed for each gene are related to their true (or mean) intensities by the following model:

xᵢⱼ=μₓᵢ+μₓᵢεₓᵢⱼ+δₓᵢⱼ, and

yᵢⱼ=μ_{yi}+μ_{yi}ε_{yij}+δ_{yij}

where (μₓᵢ,μ_{yi}) is the pair of true mean intensities for gene i. For each i and j, the multiplicative errors εₓᵢⱼ and ε_{yij}, are drawn from a bivariate normal distribution with means 0, standard deviations σ_{εx} and σ_{εy}, and correlation ρ_{ε}. The additive errors δₓᵢⱼ and δ_{yij}, are distributed analogously, with parameters σ_{δx}, σ_{δ}_{y} and ρ_{δ}. Thus, multiplicative and additive errors are independent of one another but can each be highly correlated between x and y; in practice ρ_{ε} is large and ρ_{δ} is small. While xᵢⱼ and yᵢⱼ can be negative if the foreground is less than the estimated background for a spot, the true intensities μₓᵢ and μ_{yi} must be non-negative. Consequently, the samples (xᵢⱼ and yᵢⱼ) are described by a bivariate normal probability density function p with parameters μₓᵢ and μ_{yi} σₓᵢ, σ_{yi} and ρ_{xi,yi}, where:

$${σ}_{xi} = \sqrt{{μ}_{xi}^{2} {σ}_{ϵ x}^{2} + {σ}_{δ x}^{2}}$$

$${σ}_{yi} = \sqrt{{μ}_{yi}^{2} {σ}_{ϵ y}^{2} + {σ}_{δ y}^{2}}$$

$${ρ}_{xi , yi} = \frac{{μ}_{xi} {μ}_{yi} {ρ}_{ϵ} {σ}_{ϵ x} {σ}_{ϵ y} + {ρ}_{δ} {σ}_{δ x} {σ}_{δ y}}{{σ}_{xi} {σ}_{yi}}$$

The model depends on six gene-independent parameters β=(σ_{εx}, σ_{εy}, ρ_{ε}, σ_{δx}, σ_{δy}, ρ_{δ}) and a mean pair per gene, μ=[(μₓ₁,μ_{y1}), (μₓ₂,μ_{y2}), . . . , (μ_{xN},μ_{yN}) ] for a total of 2N+6 parameters. The probability density function for gene i is p=p(xᵢⱼ, yᵢⱼ|β, μₓᵢ, μ_{yi}).

Parameter Estimation by Maximum Likelihood

Since β and μ are generally unknown, they can be estimated by using a maximum likelihood estimation (MLE) as described by Kendall and Stuart, The Advanced Theory of Statistics, Volume 2 (4^{th} ed., Macmillan Publishing Co., New York, N.Y., 1979), which is incorporated herein by reference. Likelihood functions, for gene i and over all genes, are respectively defined as:

$${L}_{i} ( β , {μ}_{xi} , {μ}_{yi} ) = {\prod }_{j = 1}^{M} P ( {x}_{ij} , {y}_{ij} / β , {μ}_{xi} , {μ}_{yi} )$$

$$L ( β , μ ) = {\prod }_{i = 1}^{N} {L}_{i} ( β , {μ}_{xi} , {μ}_{yi} )$$

The MLE parameter values maximizing L, designated β and μ, are estimates for the true parameters of the underlying statistical model. In general, these values can be found using standard optimization procedures as described by Press et al., Numerical Recipes in C: The Art of Scientific Computing (2^{nd} ed., Cambridge University Press, Cambridge, Mass.). Because N can be large β and μ, can be determined by optimizing subsets of parameters in separate stages:

(1) choose initial values for μ,

(2) select β to maximize L given current values of μ,

(3) for i=1, . . . , N: select (μₓᵢ,μ_{yi}) to maximize Lᵢ, given current values of β, and

(4) repeat (2) and (3) until β, μ have converged.

All stages of the optimization were performed using the procedure fmincon provided by Matlab and described by Coleman et al., Matlab Optimization Toolbox User's Guide (3^{rd} ed., Mathworks, Inc., Natick, Mass., 1999), which was incorporated herein by reference. The optimization was also implemented in C code, which produces comparable optimal parameters in substantially less execution time (less than 10 minutes on a Pentium III 500 for N=6000, M=4, as compared with 4-5 hours for the Matlab implementation). In both cases, all parameters converged within 250 iterations of stages (2) and (3) and are insensitive to initial choices for β and μ.

Significance Testing using Likelihood Ratios

After the parameters have been determined for a given set of observations, it is of immediate interest to use the model to identify mean intensity pairs which are significantly unequal such that μₓᵢ≈μ_{yi}, representing genes that are differentially expressed between the two cell populations. For each gene i, the generalized likelihood ratio test (GLRT) (Kendall and Stuart 1979) statistic λᵢ is computed according to:

$${λ}_{i} = - 2 ln ( \frac{{max}_{μ} {L}_{i} ( β , μ , μ )}{{max}_{{μ}_{x} , {μ}_{y}} {L}_{i} ( β , {μ}_{x} , {μ}_{y} )} )$$

Two maximizations are performed: in the numerator, the constraint μₓ=μ_{y}=μ is imposed, while in the denominator the optimization is unconstrained. Under the null hypothesis that μₓ=μ_{y}, β remains a consistent estimator when the constraint is imposed.

In the case that μₓᵢ=μ_{yi}, μᵢ follows (asymptotically in M and N) a χ² distribution with 1 degree of freedom (DOF), whereas if μₓᵢ≈μ_{yi}, the value of λᵢ is expected to be larger than would be obtained from random sampling of this distribution. To select differentially-expressed genes with a selection error of α, the false positive or Type-l error rate, one would first determine the critical value λ_{c}, for which the χ² cumulative probability distribution is equal to 1-α, then select the set of all genes i for which λᵢ is in the critical region λᵢ>λ_{c}. The particular choice of a depends on the number of genes on the array and the selection error which the individual investigator is willing to tolerate.

### EXAMPLE II

### Identification of Genes Differentially-Expressed in Response to Galactose Stimulation of Yeast Cells

This example describes application of the mathematical model of the variability observed over repeated observations of intensities for genes represented on a DNA microarray to the identification of genes differentially-expressed in response to galactose stimulation.

Assembly of the Microarray

In order to explore the performance of the test for differentially-expressed genes as shown in Example I, Saccharomyces cerevisiae cultures growing in the absence of galactose (YPR media) were compared to those growing in galactose-stimulating conditions (YPRG) using a DNA microarray of approximately 6200 nuclear yeast genes. The microarray was fabricated so as to consist of a large number of DNA spots on glass, each containing the full open-reading-frame sequence of a gene as reviewed by Lander, Nature Genetics 21: 3-4 (1999), which is incorporated herein by reference.

Initially, mRNA contained in each of two populations of cells was extracted, reverse-transcribed into cDNA, and labeled with either Cy3 or Cy5 dye as described below. Subsequently, the Cy3 and Cy5 dye preparations were combined and deposited on the microarray, where labeled molecules hybridize to the spot containing their complementary sequence.

In order to obtain the mRNA to be reverse-transcribed into cDNA, wild-type yeast (BY4741) or a congenic ga180Δ strain were inoculated in 100 ml of either galactose-inducing YPRG media (1% yeast extract, 2% peptone, 2% raffinose, 2% galactose) or non-inducing YPR media (1% yeast extract, 2% peptone, 2% raffinose). Subsequently, cultures were grown at 30° C. to a density of 1-2 OD₆₀₀, and total RNA was harvested by hot acidic phenol extraction as described by Ausubel et al., supra, (1995). Poly-A purification from total RNA was performed using Ambion Poly(A)Pure mRNA Isolation Kits (Ambion, Austin, Tex., catalogue #1915).

To assemble the DNA microarray a set of approximately 6200 known and predicted gene open reading frames from the yeast Saccharomyces cerevisiae (Research Genetics, Huntsville, Ala.) was amplified in separate 100 μL PCR reactions in a 384-well plate format. The PCR conditions were optimized depending on the length of the template, but in general were as follows: Initially 95° C. for 2 minutes; followed by 35 cycles of 94° C. for 30 seconds, 64° C. for 30 seconds and 72° C. for 2.5 minutes; and, finally, followed by 72° C. for 5 minutes. The reaction products were subsequently purified over a Sephacryl S-500 spin column (Pharmacia, Uppsala, Sweden). The purified product was then added to DMSO in a 1:1 ratio. A Molecular Dynamics Generation III microarray robotic spotter was used to print the PCR products onto 25 mm by 75 mm glass slides (Amersham, Piscataway, N.J., catalogue # RPK0328), which were subsequently spotted at 50% humidity and immediately UV cross-linked at 50 mJ of energy.

Complementary DNA synthesis and hybridization was accomplished as follows: 2 μg anchored dT25 primers and 2 μg random 9-mer primers were added to 4 μg poly-A selected mRNA and allowed to anneal at 70° C. for 5 minutes in a 12 μL volume. After 1 to 2 minutes on ice, 4 μL 5× Superscript II buffer (Gibco), 2 μL 0.1M dTT, 1 μL dNTP mix (10 mM dATP, dTTP, dGTP, and 1 mM dCTP), 1 mM of either Cy3 or Cy5 fluorescent dye (Amersham, Piscataway, N.J.), and 1 μL Superscript II reverse transcriptase were added. Reverse transcription occured at 42° C. for 2 to 2.5 hrs in the dark. Subsequently, the RNA was hydrolyzed by heating at 94° C. for 3 minutes, followed by addition of 1 μL of 5M NaOH, and incubation at 37° C. for 10 minutes. The pH was adjusted by the addition of 1 μL 5M HCl and 5 μL 1M Tris (pH 6.8) followed by cDNA purification through Millipore NAB plates (Millipore, Bedford, Mass.). Dye incorporation was assessed by measuring absorbance at 550 and 650 nm, and a sample aliquot containing about 40 pmol of dye is concentrated to less than 5 μL. Subsequent to labeling, purification, and concentration, Cy3 and Cy5 samples were combined and suspended in 40 to 45 μL of hybridization solution containing 50% formamide, 5× Denhardt's solution, 5× SSC and 0.1% SDS. The hybridization mixture was subsequently applied to the array slide beneath a coverslip and allowed to incubate in a sealed, humid chamber overnight for 16 to 18 hours at 42° C. The slide was then washed in 2× SSC/0.1% SDS for 5 minutes at 42° C., followed by a 5 minute wash in 0.1× SSC/0.1% SDS for 5 minutes at room temperature and, finally, two additional washes in 0.1× SSC, each for two minutes. The slide was rinsed briefly in distilled water and immediately dried with compressed air. After hybridization and washing, the array slides were scanned using a scanning laser fluorescence microscope (Molecular Dynamics Generation III Scanner, Molecular Dynamics, Sunnyvale, Calif.).

Each gene was represented by two spots located on opposite sides of the array. A total of four (x,y) intensity pairs was obtained for each gene by performing replicate hybridizations to two of the above microarrays (N=6200, M=4), with x and y representing intensities in YPR and YPRG respectively. In the first hybridization, RNA from the YPR condition was labeled with Cy3 dye, while RNA from the YPRG condition was labeled with Cy5 dye; in the second hybridization the reverse labeling scheme was used. The β and μ values were determined for these data using our maximum likelihood approach, and the λ₁ statistic was computed for each gene. Values for β were as follows: 0.367, 0.391, 0.862, 89.6, 339.0, 0.319.

In order to determine a reasonable choice for the critical value λ_{c} used to select differentially-expressed genes, a series of control experiments was performed in which two cell populations were cultured separately using identical strains and YPRG growth conditions. These two populations were compared as described before by obtaining a total of M=4 repeat samples per gene and determining values of β, μ and λ. In general, these control data had fewer large values of λ than did the YPR versus YPRG data, and followed a χ² distribution as determined by a q-q plot. However, both data sets had significantly larger values of λ than expected for a χ² with 1 DOF. This can be due to the small-sample bias of maximum likelihood methods, resulting in λᵢ, resulting in λᵢ statistics that are not χ² with 1 DOF even if μₓᵢ=μ_{yi}, for all i.

We chose λ_{c}=25.7, the value at which less than 0.1% of genes (approximately 6 out of 6200) would be in the critical region in the control experiment. This value was then applied to select differentially-expressed genes from the YPR versus YPRG data.

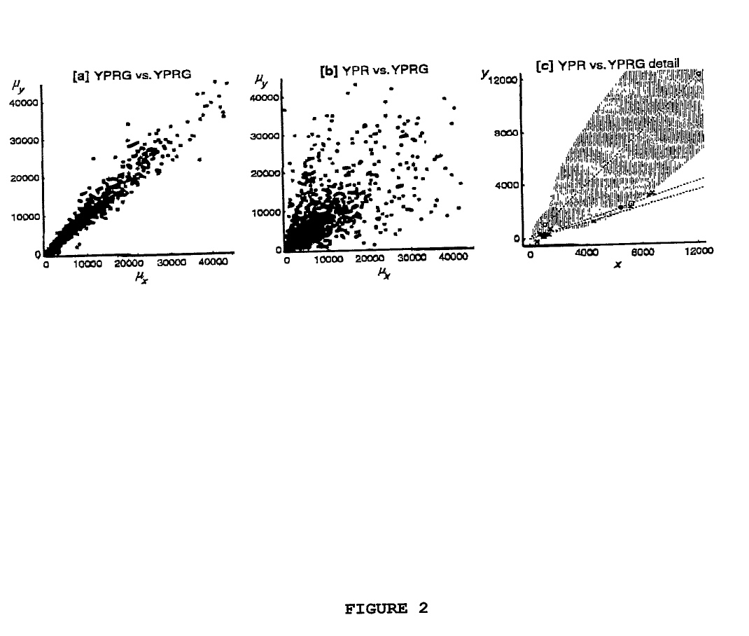

FIGS. 2A and 2B show scatter plots of estimated μ_{y} versus μₓ values for each gene for the control experiment and the YPR versus YPRG experiment, respectively. The most highly significant genes out of a total of 555 selected as significant are shown in Table 1. The values shown in Table 1 are in good agreement with previous experimental evidence with the galactose-induction pathway structural genes GAL1, GAL7 and GAL10 appearing as the top three most significant differentially-expressed genes.

TABLE 1
Genes Differentially Expressed Between
Galactose Non-Inducing (YPR) and Inducing (YPRG) Conditions.

| GENE | ROLE | λ | μₓ | μ_{y} | μₓ/μ_{y} |
|---|---|---|---|---|---|
| GAL1 | galactose | 95.4 | 145 | 110644 | 766 |
| | metabolism | | | | |
| GAL10 | galactose | 88.1 | 109 | 36656 | 338 |
| | metabolism | | | | |
| Gal7 | galactose | 86.7 | 59 | 76849 | 1300 |
| | metabolism | | | | |
| YNL194C | unknown | 75.0 | 18533 | 1360 | 0.073 |
| JEN1 | transport | 72.2 | 21124 | 889 | 0.042 |
| YNL195C | unknown | 72.0 | 7639 | 710 | 0.093 |
| ALD6 | ethanol utilization | 71.5 | 9774 | 517 | 0.053 |
| RHR2 | glycerol metabolism | 71.1 | 1181 | 22586 | 19 |
| YMR318C | unknown | 69.1 | 2457 | 29930 | 12 |
| HSP26 | diauxic shift | 68.1 | 71988 | 11435 | 0.16 |

In the scatter plots shown in FIG. 2, genes with λ₁>25.7 have significantly different μ_{y} and 82 ₓ and are shown in red. To show detail, axes limits are truncated to 45000: the maximum (μₓ,μ_{y}) observed was (1.8×10⁵, 1.4×10⁵).

FIG. 2(C) shows the distribution of four (x,y) pairs for two genes in the YPR versus YPRG comparison. Samples for each gene are denoted by red or black crosses respectively, with corresponding averages (<x>,<y>) denoted by squares and MLE-estimated means (μₓ,μ_{y}) denoted by filled circles. Open circles represent the estimated means under the added constraint μₓ=μ_{y} Pink and gray ellipses define regions containing 95% of the error model probability distribution at these constrained means for the red and black-colored genes, respectively. Dotted lines of constant ratio, drawn through the origin and each constrained and unconstrained (μₓ,μ_{y}) pair, are shown for reference. In FIG. 2C, although the genes have similar average expression ratios <x>/<y> (2.9 versus 3.5 for the red versus black-colored gene), the red-colored gene was significant by the likelihood test (λ>37.4). The black-colored gene was not significant (λ=13.8), due to its compatibility with the constrained error model. The difference in λ arises because the samples corresponding to the red-colored gene are higher in intensity than the samples corresponding to the black-colored gene.

As described in Example I, equation 5 computes λ for each gene by optimizing the model parameters (μₓ, and μ_{y}) with and without the constraint μₓ=μ_{y}, and subsequently compares the likelihood of the (x,y) samples under the constrained and unconstrained models. As represented by the pink ellipse shown in FIG. 2C, the four red-colored samples are in the tail of the probability distribution for the error model with the constraint imposed, resulting in a reduced likelihood L and thus a relatively high significance value 1. In contrast, as represented by the grey ellipse shown in FIG. 2C, the black-colored samples are relatively well explained by the constrained error model distribution, resulting in a lower value of λ. Notably, if the ratio statistic r were applied with the commonly-used threshold r_{c}=3.0, the black gene would be accepted as significant while the red gene would not.

Effect of Sample Size on Parameter Estimates

The more genes and samples per gene are available, the more accurate the estimates of β and μ. To determine the efficacy of parameter estimation, representative parameters βₛᵢₘ and μₛᵢₘ were used to randomly simulate data sets of M samples over N genes according to the error model equations (2) and (3) disclosed in Example I. Values for β and μ were estimated for each data set and the resulting distribution of β over 30 simulations was characterized by parameter means <β> and standard deviations βₛ. In simulations with M=50, N=100, parameter estimates were tightly distributed around their true values such that <β>=βₛᵢₘ±2% and s_{β}≦(0.3)<β> for all parameters β. In contrast, for very small data sets with M=4, N=100 these estimates were highly variable over the 30 simulations (s_{β}≦(0.74)<β>) and biased: βₛᵢₘ was under- or overestimated by 5 percent to 50 percent across the six parameters of <i>. In order to more closely model experiments performed with a yeast microarray, simulations with M=4, N=6000 were also examined. Estimates were generally biased but this bias was smaller (<β>=βₛᵢₘ±25%) and the variability of estimation also was less (s_{β}≦(0.05)<β>). Thus, with regard to parameter estimation, a large number of genes appears to at least partially compensate for the destabilizing effect of a small number of repeats.

To further study the effect of sample size on significance testing in the YPR versus YPRG study, β, μ and λ were determined using just two of the available four samples per gene by drawing one spot per gene over the two replicate hybridizations. In this case, the number of genes selected as differentially-expressed was less, 227 as compared to 555 using λ₁>25.7, although 85 percent of these genes were previously identified as significant when using four samples per gene. The genes GAL1, GAL7, and GAL10 also were identified as significant, but were no longer among the top ten with largest λ. While these genes still had a very extreme expression ratio (μ_{y}/μₓ) their intensity samples were by chance more variable than those of other genes with extreme expression ratios and thus their corresponding value of λ was smaller.

Ratios of Intensity are Approximately Equal to Ratios of Hybridized cDNA

Although the proposed method identifies genes having different mean intensities μₓᵢ and μ_{yi}, in order to conclude that these genes are differentially-expressed, intensity differences or ratios must be at least approximately proportional to differences in RNA copy number per cell. Since it is expected that either low or high copy number could lead to saturation in the measured intensity, a series of controlled experiments was performed to determine whether this relationship is linear over a reasonable range of copy number.

First, a mixture of ga180Δ cDNA was created by extracting mRNA from yeast with a complete deletion of the GAL80 gene, which was labelled with Cy3 and Cy5 dyes in separate reactions, and subsequently combining the reactions into one tube. The mixture was hybridized to a yeast genome microarray, and the resulting image checked to ensure that intensity was not detectable above background for spots representing GAL80 and that all spots had roughly equal Cy3 and Cy5 intensities. Next, Cy3- and Cy5-labeled DNA sequences corresponding to the GAL80 open reading frame were added to the ga180Δ cDNA mixture at fixed molar ratios of Cy3:Cy5 dye.

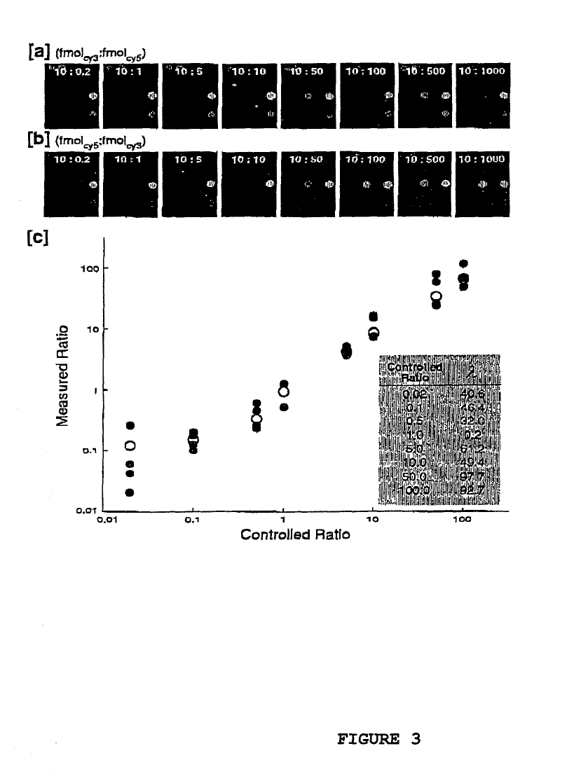

As shown in FIGS. 3A and 3B, array hybridizations were performed for each of eight controlled GAL80 ratios. Data sets consisting of four (x,y) intensity measurements per gene were obtained at each controlled GAL80 ratio by using two spots from a forward Cy3:Cy5 labeling scheme and two spots from a reverse Cy5:Cy3 labeling scheme. Parameters β and μ were determined separately for each data set, and the corresponding measured ratio for GAL80 was defined as μ_{y}/μₓ.

FIG. 3C shows a scatter plot of each measured ratio versus controlled ratio for the forward-array (red dots) or reverse-array (green dots) and demonstrates that, while saturation occurs at the lower extreme, the system is approximately linear over a range of 3 orders of magnitude. The ratio of estimated means μ_{y,}/μₓ also is shown and denoted by open circles. The inset table shows the value of λ for the GAL80 gene in each of the eight controlled ratios. The ratio of estimated means μ_{y,}/μₓ is denoted by open circles. The inset table shows the value of λ for the GAL80 gene in each of the eight controlled ratios. Except where the controlled ratio was equal to one, all measured GAL80 ratios had λ>25.7 and thus were differentially-expressed by the likelihood test.

At the upper end of the investigated range, GAL80 was added at 1000 fmol and measured at 32,436 intensity units as averaged over four samples. Only 14 genes on the array had higher intensities, the two largest being TDH3 (81255 units) and EN02 (55766 units). At the lower end of the range, GAL80 was added at 0.2 fmol and measured at 284 units: approximately 1000 genes had lower intensities. These genes are either not expressed or are beneath the range of detection.

The intensities of several genes whose RNA copy number per cell has been determined experimentally also were determined (Iyer and Struhl, Proc. Natl. Accad. Sci. USA 93:5208-5212 (1996)). The RNA corresponding to the TRP3 gene has been observed at 1.9 copies per cell in YPR media, and had a corresponding average intensity of 597 (standard deviation of 259) in the YPR condition of the YPR versus YPRG array experiment. In contrast, GAL1 mRNA is present at less than <0.1 copies per cell in YPR and was not significantly above background intensity on our yeast array. Thus, most yeast genes, approximately 4000 to 5000, appear to have intensities within the linear range of the microarray system and the lower limit of detection is between 0.1 and 1.9 copies/cell.

Application of the Likelihood Model to Compare and Contrast Parameters over Different Types of Repeat Measurements

A test microarray having 96 genes spotted 16 times each was constructed to use the error model to compare the combined variability present across an entire experiment to that introduced during array hybridization and quantitation alone. Ten cultures were grown involving identical strains and YPRG conditions, independently in separate containers, and RNA prepared from each of the ten cultures. Five of the preparations were labeled using Cy3, while the remaining five were labeled using Cy5. The mixtures were combined in Cy3-Cy5 pairs, and each of the five pairs hybridized to separate test arrays. Two types of data sets were drawn from these experiments. In the first type of data set, repeats were drawn from the 16 replicate spots per gene on a single array (within-slide data, N=96, M=16).

Parameters were estimated by maximum likelihood, independently for data sets formed using each of the five test arrays. Mean and standard deviation values over the estimates are shown in Row 1 of Table 2. In the second type of data set, repeats were drawn from a single spot of each gene on the array over the five hybridizations to separate test arrays (between-slide data, N=96, M=5). In this case, parameters β were estimated 16 times, separately for data sets formed using each of the 16 spots per gene available on the array (see Table 2, Row 2). Although the multiplicative errors εₓ and ε_{y} have nearly identical standard deviations for the within- and between-slide repeats, they are considerably more correlated within a slide than between slides. In addition, the within-slide measurements have less variability with regard to the additive error components δₓ and δ_{y}.

TABLE 2
Comparison of Error Model Parameters for Five
Within-Slide and 16 Between-Slide Data Sets.

| Source of | | | | | |
|---|---|---|---|---|---|
| Variation | σ_{εx} | σ_{εy} | ρ_{ε} | σ_{δx} | σ_{δy} |
| within slide | 0.35 | 0.306 | 0.981 | 251 | 374 |
| mean | (.063) | (.061) | (.0069) | (49) | (105) |
| standard error | | | | | |
| between slides | 0.365 | 0.315 | 0.967 | 422 | 569 |
| mean | (.0084) | (.0073) | (.0017) | (12) | (13) |
| standard error | | | | | |

For these optimizations, the parameter ρ_{δ} did not always converge: it was therefore set to zero during parameter estimation and does not appear in Table 2. In comparison with other data sets, the prenormalized x′ and y′ intensities of all 96 genes in the test data were moderate to relatively high. Therefore, ρ_{δ} was likely ill-determined because under the error model, ρ_{δ} is dominated by ρ_{ε} for larger intensities.

Although the invention has been described with reference to the disclosed embodiments, those skilled in the art will readily appreciate that the specific experiments detailed are only illustrative of the invention. It should be understood that various modifications can be made without departing from that spirit of the invention. Accordingly, the invention is limited only by the following claims.

We claim:

1. A method of determining a true signal of an analyte, comprising:
(a) measuring an observed signal x for one or more analytes, and
(b) determining a mean signal (μ) and a system parameter (β) for said analyte that produce enhanced values for a probability likelihood of said observed signal, said observed signal being related to said mean signal by an additive error (δ) and a multiplicative error (ε), wherein said system parameter specifies properties of said additive error (δ) and said multiplicative error (ε).

2. The method of claim 1, further comprising selecting a mean signal μ that provides a maximum probability of likelihood given said observed signal.

3. The method of claim 1, wherein said additive and multiplicative errors are independent with respect to each other.

4. The method of claim 1, wherein said observed signal and said mean signal further comprises the relationship;
xᵢⱼ=μₓᵢ+μₓᵢεₓᵢⱼ+δₓᵢⱼ,
where each measurement j=1, . . . , M, each analyte i=1, . . . , N, and where xᵢⱼ is the observed signal and μₓᵢ is the mean signal.

5. The method of claim 1, wherein said additive and multiplicative errors further comprise a univariate distribution.

6. The method of claim 5, wherein said univariate distribution is a parametric distribution.

7. The method of claim 6, wherein said parametric distribution is a univariate normal distribution.

8. The method of claim 7, wherein said univariate normal distribution and said system parameter further comprise a multiplicative error term consisting of a normal distribution having standard deviation with respect to a signal mean (σ_{εx}) and an additive error term consisting of a normal distribution having standard deviation with respect to a signal mean (σ_{δx}).

9. The method of claim 6, wherein said parametric distribution is a t-distribution.

10. The method of claim 6, wherein said parametric distribution is a gamma distribution.

11. The method of claim 1, wherein said mean signal and system parameter are determined at the same time.

12. The method of claim 1, wherein said system parameter is determined before said mean signal is determined.

13. The method of claim 12, wherein said predetermined system parameter is used to determine said mean signal.

14. The method of claim 1, wherein said enhanced values for said probability likelihood of said observed signals are produced one or more times until said mean signal and said system parameter converge.

15. The method of claim 1, wherein said mean signal and said system parameter are determined by a method selected from the group consisting of maximum likelihood estimation (MLE), Quasi-Maximum Likelihood and Generalized Method of Moments.

16. The method of claim 1, wherein determining said mean signal and said system parameter further comprises a non-linear optimization algorithm.

17. The method of claim 16, wherein said optimization algorithm is selected from the gorup consisting of Gradient Descent, Newton-Raphson and Simulated Annealing.

18. A method of determining a true signal of an analyte, comprising:
(a) obtaining an observed signal x for one or more analytes;
(b) providing a mean signal (μ) and a system parameter (β) for said analyte;
(c) computing a probability likelihood of said observed signal, said observed signal being related to said mean signal by an additive error (δ) and a multiplicative error (ε), where said system parameter specifies properties of said additive error and said multiplicative error, and
(d) selecting a mean signal μ and a system parameter (β) that provides a maximum probability likelihood of occurrence given said observed signal.

19. The method of claim 18, wherein said additive and multiplicative errors are independent with respect to each other.

20. The method of claim 18, wherein said observed signal and said mean signal further comprises the relationship:
xᵢⱼ=μₓᵢ+μₓᵢεₓᵢⱼ+δₓᵢⱼ,
where each measurement j=1, . . . , N, each analyte i=1, . . . , N, and where xᵢⱼ is the observed signal and μₓᵢ is the mean signal.

21. The method of claim 18, wherein said additive and multiplicative errors further comprise a univariate distribution.

22. The method of claim 1, wherein said univariate distribution is a parametric distribution.

23. The method of claim 22, wherein said parametric distribution is a univariate normal distribution.

24. The method of claim 23, wherein said univariate normal distribution and said system parameter further comprise a multiplicative error term consisting of a normal distribution having standard deviation with respect to a signal mean (σ_{εx}), and an additive error term consisting of a normal distribution having standard deviation with respect to a signal mean (σ_{δx}).

25. The method of claim 22, wherein said parametric distribution is a t-distribution.

26. The method of claim 22, wherein said parametric distribution is a gamma distribution.

27. The method of claim 18, wherein said mean signal and system parameter are selected at the same time.

28. The method of claim 18, wherein said system parameter is selected before said mean signal is determined.

29. The method of claim 28, wherein said preselected system parameter is used to select said mean signal.

30. The method of claim 18, further comprising computing said probability likelihood one or more times until said mean signal and said system parameter converge.

31. The method of claim 18, wherein said mean signal and said system parameter are determined by a method selected from the group consisting of maximum likelihood estimation (MLE), Quasi-Maximum Likelihood and Generalized Method of Moments.

32. The method of claim 18, wherein selecting said mean signal and said system parameter further comprises a non-linear optimization algorithm.

33. The method of claim 32, wherein said optimization algorithm is selected from the group consisting of Gradient Descent, Newton-Raphson and Simulated Annealing.

34. A method of determining relative amounts of an analyte between samples, comprising:
(a) measuring observed signals x and y for an analyte within two or more sample pairs, and
(b) determining a mean signal pair per analyte (μ) and a system parameter (β) for each sample pair that produce enhanced values for a probability likelihood of said observed signals, said observed signals being related to said mean signals by an additive error (δ) and a multiplicative error (ε), wherein said system parameter specifies properties of said additive error (δ) and said multiplicative error (ε).

35. The method of claim 34, further comprising selecting a mean signal p that provides a maximum probability of occurrence given said observed signals.

36. The method of claim 34, wherein said additive and multiplicative errors are independent with respect to each other.

37. The method of claim 34, wherein said observed signals and said mean signal pair per analyte within said sample pairs further comprise the relationship:
xᵢⱼ=μₓᵢ+μₓᵢεₓᵢⱼ+δₓᵢⱼ, and
yᵢⱼ=μ_{yi}+μ_{yi}ε_{yij}+δ_{yij}
where each measurement j equals 1 through M and each analyte i equals 1 through N; where xᵢⱼ and yᵢⱼ are the observed signals, and where μₓᵢ and μ_{yi} are the mean signals.

38. The method of claim 34, wherein said additive and multiplicative errors further comprise a bivariate distribution.

39. The method of claim 38, wherein said bivariate distribution is a parametric distribution.

40. The method of claim 38, wherein said parametric distribution is a bivariate normal distribution.

41. The method of claim 40, wherein said bivariate normal distribution and said system parameter further comprises a multiplicative error term consisting of a standard deviation with respect to a mean of signal x (σ_{εx}), a standard deviation with respect to a mean of signal y (σ_{εy}) and a correlation between signals x and y (ρ_{ε}), and an additive error term consisting of a standard deviation with respect to a mean of signal x (σ_{δx}), a standard deviation with respect to a mean of signal y (σ_{δx}) and a correlation between signals x and y (ρ_{δ}).

42. The method of claim 39, wherein said parametric distribution is a t-distribution.

43. The method of claim 39, wherein said parametric distribution is a bivariate gamma distribution.

44. The method of claim 34, wherein said mean signal pair per analyte and system parameter are determined at the same time.

45. The method of claim 34, wherein said system parameter is determined before said mean signal pair per analyte is determined.

46. The method of claim 45, wherein said predetermined system parameter is used to determine said mean signal pair per analyte.

47. The method of claim 34, wherein said enhanced values for said probability likelihood of said observed signals are produced one or more times until said mean signal pair per analyte and said system parameter converge.

48. The method of claim 34, wherein determining said mean signal pair per analyte and said system parameter further comprises a non-linear optimization algorithm.

49. The method of claim 48, wherein said optimization algorithm is selected from the group consisting of Gradient Descent, Newton-Raphson and Simulated Annealing.

50. The method of claim 34, further comprising identifying significantly unequal mean signal pairs per analyte by a statistical difference indicator.

51. The method of claim 50, wherein said difference indicator further comprises a generalized likelihood ratio test statistic (λ).

52. A method of determining relative amounts of an analyte between samples, comprising:
(a) obtaining observed signals x and y for an analyte within two or more sample pairs;
(b) providing a mean signal pair per analyte (μ) and a system parameter (β) for each sample pair;
(c) computing a probability likelihood of said observed signals, said observed signals being related to said mean signal by an additive error (δ) and a multiplicative error (ε), where said system parameter specifies the properties of said additive error and said multiplicative error, and
(d) selecting a mean signal p and a system parameter (β) that provides a maximum probability likelihood of occurrence given said observed signals.

53. The method of claim 52, wherein said additive and multiplicative errors are independent with respect to each other.

54. The method of claim 52, wherein said observed signals and said mean signal pair per analyte within said sample pairs further comprise the relationship:
xᵢⱼ=μₓᵢ+μₓᵢεₓᵢⱼ+δₓᵢⱼ, and
yᵢⱼ=μ_{yi}+μ_{yi}ε_{yij}+δ_{yij}
where each measurement j equals 1 through M and each analyte i equals 1 through N; where xᵢⱼ and yᵢⱼ are the observed signals, and where μₓᵢ and μ_{yi} are the mean signals.

55. The method of claim 52, wherein said additive and multiplicative errors further comprise a bivariate distribution.

56. The method of claim 55, wherein said bivariate distribution is a parametric distribution.

57. The method of claim 56, wherein said parametric distribution is a bivariate normal distribution.

58. The method of claim 57, wherein said bivariate normal distribution and said system parameter further comprise a multiplicative error term consisting of a standard deviation with respect to a mean of signal x (σ_{εx}), a standard deviation with respect to a mean of signal y (σ_{εy}) and a correlation between signals x and y (ρ_{ε}), and an additive error term consisting of a standard deviation with respect to a mean of signal x (σ_{δx}), a standard deviation with respect to a mean of signal y (σ_{δy}) and a correlation between signals x and y (ρ_{δ}).

59. The method of claim 56, wherein said parametric distribution is a t-distribution.

60. The method of claim 56, wherein said mean signal pair per analyte and system parameter are determined at the same time.

61. The method of claim 52, wherein said system parameter is determined before said mean signal pair per analyte is determined.

62. The method of claim 61, wherein said predetermined system parameter is used to determine said mean signal pair per analyte.

63. The method of claim 52, further comprising computing said probability likelihood of said observed signals one or more times until said mean signal pair per analyte and said system parameter converge.

64. The method of claim 52, wherein said mean signal pair per analyte and said system parameter are determined by a method selected from the group consisting of maximum likelihood estimation (MLE), Quasi-Maximum Likelihood and Generalized Method of Moments.

65. The method of claim 52, wherein selecting said mean signal pair per analyte and said system parameter further comprises a non-linear optimization algorithm.

66. The method of claim 65, wherein said optimization algorithm is selected form the group consisting of Gradient Descent, Newton-Raphson and Simulated Annealing.

67. The method of claim 52, further comprising identifying said mean signal pair per analyte that are significantly unequal using a difference indicator.

68. The method of claim 67, wherein said difference indicator further comprises a generalized likelihood ratio test statistic (λ).

69. The method of claim 67, further comprising selecting two or more mean signal pairs per analyte having a difference indicator greater than that corresponding to a false positive error rate.

70. The method of claim 52, wherein said analyte is a nucleic acid or polypeptide.

71. A method of determining relative amounts of analytes between samples, comprising:
(a) obtaining observed signals x and y for a plurality of immobilized analytes within two or more sample pairs;
(b) determining a mean signal pair per analyte (μ) and a system parameter (β) for each sample pair that provides a maximum probability likelihood of occurrence given said observed signals, said observed signals being related to said mean signal by an additive error (δ) and a multiplicative error (ε), where said system parameter specifies the properties of said additive error and said multiplicative error, and
(c) identifying one or more mean signal pairs per analyte that is significantly unequal.

72. The method of claim 71, wherein said additive and multiplicative errors are independent with respect to each other.

73. The method of claim 71, wherein said observed signals and said mean signal pair per analyte within said sample pairs further comprise the relationship:
xᵢⱼ=μₓᵢ+μₓᵢεₓᵢⱼ+δₓᵢⱼ, and
yᵢⱼ=μ_{yi}+μ_{yi}ε_{yij}+δ_{yij}
where each measurement j equals 1 through M and each analyte i equals 1 through N; where xᵢⱼ and yᵢⱼ are the observed signals, and where μₓᵢ and μ_{yi} are the mean signals.

74. The method of claim 71, wherein said one or more mean signal pairs per analyte are identified as significantly unequal by using a difference indicator.

75. The method of claim 74, wherein said difference indicator further comprises a generalized likelihood ratio test statistic (λ).

76. The method of claim 74, further comprising selecting two or more mean signal pairs per analyte having a difference indicator greater than that corresponding to a false positive error rate.

77. The method of claim 71, wherein said analyte is a nucleic acid or polypeptide.

78. The method of claim 71, wherein said plurality of analytes further comprises about 1,000 or more different analytes.

79. The method of claim 71, wherein said plurality of analytes further comprises about 10,000 or more different analytes.

80. The method of claim 71, wherein said plurality of analytes further comprises about 30,000 or more different analytes.

81. The method of claim 71, further comprising analytes mobilized on a microarray.

82. The method of claim 71, further comprising the steps of:
(a) obtaining one or more reference signals, and
(b) determining a mean signal pair (μ) and a system parameter (β) for a sample pair comprising said observed signal x or y and said reference signal that provides a maximum probability likelihood of occurrence given said reference and observed signals, said reference and observed signals being related to said mean signal by an additive error (δ) and a multiplicative error (ε), wherein said system parameter specifies the properties of said additive error and said multiplicative error.

83. A method of determining relative amounts of an analyte between samples, comprising:
(a) obtaining a reference signal;
(b) obtaining observed signals x and y for an analyte within two or more sample pairs;
(c) determining system parameters (β₁, β₂) for a sample pair comprising said observed signals x or y and said reference signal that provide a probability likelihood of said occurrence given said observed and reference signals, said observed and reference signals being related to said mean signal by an additive error (δ) and a multiplicative error (ε), where said system parameter specifies the properties of said additive error and said multiplicative error;
(d) determining mean signal pairs (μ₁, μ₂) for said sample pair comprising maximizing a product of terms for said probability likelihood of said sample pair of observed signals x or y and said reference signal for said analyte, and
(e) selecting a mean signal μₓ or μ_{y} that provides a maximum probability likelihood of occurrence given said observed signals and system parameters β₁ and β₂.

84. The method of claim 83, wherein said mean signal pairs (μ₁, μ₂) are determined using β₁ and β₂ obtained from step (c).

85. A method of determining relative amounts of an analyte between samples, comprising:
(a) measuring observed signals x, y and z for an analyte within two or more sample sets, and
(b) determining a mean signal set per analyte (μ) and a system parameter (β) for each sample set that produce enhanced values for a probability likelihood for said observed signals, said observed signals being related to mean signals by an additive error (δ) and a multiplicative error (ε).
